(19) United States
(12) Patent Application Publication
(10) Pub. No.: US 2025/0064374 A1
(43) Pub. Date: Feb. 27, 2025

(54) ELECTROCARDIOGRAM EVALUATION DEVICE, ELECTROCARDIOGRAM EVALUATION METHOD, AND STORAGE MEDIUM

(71) Applicant: NEC Corporation, Minato-ku, Tokyo (JP)

(72) Inventors: Yuan LUO, Tokyo (JP); Kosuke NISHIHARA, Tokyo (JP); Osamu HISAMATSU, Tokyo (JP); Mitsuru NOMA, Tokyo (JP)

(73) Assignee: NEC Corporation, Minato-ku, Tokyo (JP)

(21) Appl. No.: 18/725,854

(22) PCT Filed: Jan. 7, 2022
(86) PCT No.: PCT/JP2022/000351

§ 371 Date: Jul. 1, 2024

(51) Int. Cl. A61B 5/361; A61B 5/00; A61B 5/339

(52) U.S. Cl. CPC A61B 5/361; A61B 5/339; A61B 5/7267

(57) ABSTRACT

The electrocardiogram evaluation device 1X mainly includes an acquisition means 30X, a selection means 31X, and an evaluation means 33X. The acquisition means 30X is configured to acquire electrocardiogram data regarding an electrocardiogram of a subject. The selection means 31X is configured to select, from the electrocardiogram data, lead data of one or more leads corresponding to a target disease of examination. The evaluation means 33X is configured to evaluate the electrocardiogram regarding the target disease based on the selected lead data.

### TECHNICAL FIELD

The present disclosure relates to the technical fields of an electrocardiogram evaluation device, an electrocardiogram evaluation method, and a storage medium for evaluating an electrocardiogram as to the presence or absence of a disease.

### BACKGROUND

There is a device configured to determine the presence or absence of a disorder of a subject by analyzing the measured electrocardiogram of the subject. For example, Patent Literature 1 discloses an electrocardiogram analysis device configured to analyze an electrocardiogram waveform measured from a subject with reference to a predetermined arrhythmia criterion and output an alarm or the like if an arrhythmia is detected.

### CITATION LIST

### Patent Literature

Patent Literature 1: JP 2014-150826A

### SUMMARY

### Problem to be Solved

When evaluating the presence or absence of a disease based on an electrocardiogram, usage of lead data which is not related to a disease of interest could lead to the deterioration of the accuracy.

In view of the issues described above, one object of the present invention is to provide an electrocardiogram evaluation device, an electrocardiogram evaluation method, and a storage medium capable of accurately conducting an evaluation of an electrocardiogram of a subject related to a disease of interest.

### Means for Solving the Problem

In one mode of the electrocardiogram evaluation device, there is provided an electrocardiogram evaluation device including:
an acquisition means configured to acquire electrocardiogram data regarding an electrocardiogram of a subject;
a selection means configured to select, from the electrocardiogram data, lead data of one or more leads corresponding to a target disease of examination; and
an evaluation means configured to evaluate the electrocardiogram regarding the target disease based on the selected lead data.

In one mode of the electrocardiogram evaluation method, there is provided an electrocardiogram evaluation method executed by the computer, the electrocardiogram evaluation method including:
acquiring electrocardiogram data regarding an electrocardiogram of a subject;
selecting, from the electrocardiogram data, lead data of one or more leads corresponding to a target disease of examination; and
evaluating the electrocardiogram regarding the target disease based on the selected lead data.

In one mode of the storage medium, there is provided a storage medium storing a program executed by a computer, the program causing the computer to:
acquire electrocardiogram data regarding an electrocardiogram of a subject;
select, from the electrocardiogram data, lead data of one or more leads corresponding to a target disease of examination; and
evaluate the electrocardiogram regarding the target disease based on the selected lead data.

### Effect

An example advantage according to the present invention is to accurately conduct an evaluation of an electrocardiogram of a subject relating to a disease of interest.

### BRIEF DESCRIPTION OF THE DRAWINGS

FIG. 1 It illustrates an example of a hardware configuration of an electrocardiogram evaluation device.

FIG. 2 It illustrates a functional block diagram of the electrocardiogram evaluation device.

FIG. 3 It illustrates the processing flow in the case that the disease of interest is atrial fibrillation.

FIG. 4 It illustrates an example of an electrocardiogram evaluation screen image.

FIG. 5 It is an example of a flowchart showing an outline of a process executed by the electrocardiogram evaluation device in a first example embodiment.

FIG. 6 It is a functional block diagram of the electrocardiogram evaluation device in a second example embodiment.

FIG. 7 It is an enlarged view of the waveform of the lead data.

FIG. 8 It is an example of a flowchart showing an outline of a process executed by the electrocardiogram evaluation device in the second example embodiment.

FIG. 9 It is a block diagram of an electrocardiogram evaluation device in a third example embodiment.

FIG. 10 It is an example of a flowchart showing a processing procedure of the electrocardiogram evaluation device in the third example embodiment.

### EXAMPLE EMBODIMENTS

Hereinafter, example embodiments of an electrocardiogram evaluation device, an electrocardiogram evaluation method, and a storage medium will be described with reference to the drawings.

### First Example Embodiment

### (1) System Configuration

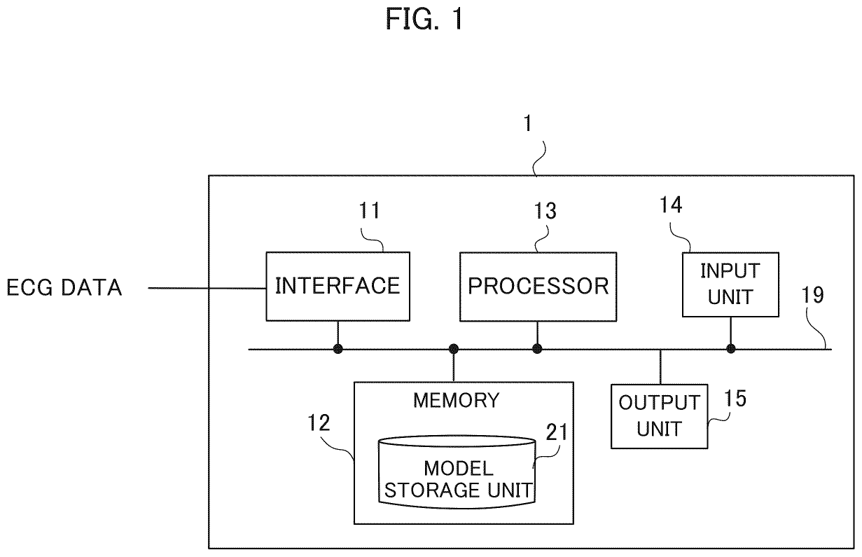

FIG. 1 shows an example of a hardware configuration of an electrocardiogram evaluation device 1. The electrocardiogram evaluation device 1 analyzes an electrocardiogram measured from a subject to evaluate the electrocardiogram related to a target disease (also referred to as “target disease”) of diagnosis. Then, the electrocardiogram evaluation device 1 mainly includes an interface 11, a memory 12, a processor 13, an input unit 14, and an output unit 15. Each of these elements is connected to one another via a data bus 19. The target disease is mainly assumed herein to be a circulatory disease, but may be any disease detectable by electrocardiogram analysis.

The interface 11 performs an interface operation between the electrocardiogram evaluation device 1 and an external device. In this instance, the interface 11 is a communication interface such as a network adapter for performing communication with an external device that is a device separate from the electrocardiogram evaluation device 1 by wire or wirelessly, or a hardware interface conforming to USB (Universal Serial Bus), SATA (Serial AT Attachment), or the like.

In the present example embodiment, the interface 11 receives ECG data, which is data related to an electrocardiogram of the subject, from an external device and supplies the ECG data to the processor 13. In this case, the external device may be an electrocardiogram measuring device that measures the electrocardiogram from the electrocardiogram signal of the electrodes put on the subject, or may be a device that stores the measurement results outputted by the electrocardiogram measuring device. The ECG data is data indicating measurement results on the electrocardiogram of the subject. In this example embodiment, the ECG data is assumed to be data of the twelve-lead electrocardiogram obtained through an examination for recording the electrical activity and change in the heart from ten electrodes in total attached to six points of the chest, both wrists, and both ankles. The ECG data may be raw data of an electrocardiogram of the subject outputted by the electrocardiogram measuring device, or may be a file of an electrocardiogram (e.g., an electrocardiogram image or a PDF file) converted into a predetermined format.

The memory 12 is configured by a variety of volatile memories, which are used as a working memory, and non-volatile memories, which store the information required for the electrocardiogram evaluation device 1 to process data, such as a RAM (Random Access Memory) and a ROM (Read Only Memory). The memory 12 may include an external storage device such as a hard disk connected to or incorporated into the electrocardiogram evaluation device 1 or may include a storage medium such as a removable flash memory. The memory 12 stores a program for the electrocardiogram evaluation device 1 to execute each process in the present example embodiment.

The memory 12 functionally includes a model storage unit 21. The model storage unit 21 stores the parameters of the model configured to output, based on the lead data representing each lead of the twelve-lead electrocardiogram, the evaluation result indicating the presence or absence of the target disease. For example, the model is a machine learning model such as a neural network and a support vector machine, and is trained in advance to output an evaluation result regarding the presence or absence of the target disease when lead data is inputted to the model, wherein the learned parameters are stored in the model storage unit 21. If the model is configured by the neural network, the model storage unit 21 stores various parameters such as a layer structure, a neuron structure of each layer, the number of filters and the size of filters in each layer, and the weight for each element of each filter, for example.

The model may be a model trained for each lead. In this case, the model storage unit 21 stores the parameters of the model for each lead. In another example, the model may be a model trained for each target disease. In this case, the model storage unit 21 stores the parameters of respective models corresponding to possible target diseases.

The processor 13 executes a predetermined process by executing a program or the like stored in the memory 12. Examples of the processor 13 is a processor such as a CPU (Central Processing Unit), a GPU (Graphics Processing Unit), and a TPU (Tensor Processing Unit). The processor 13 may be configured by a plurality of processors. The processor 13 is an example of a computer.

The input unit 14 generates an input signal. Examples of the input unit 14 include a button, a touch panel, a remote controller, and a voice input device. The output 15 displays and/or outputs, by audio, information under the control of the processor 13. Examples of the output unit 15 include a display, a projector, and a speaker. The output unit 15 is an example of a display device.

The configuration of the electrocardiogram evaluation device 1 shown in FIG. 1 is an example, and various changes may be made thereto. For example, the ECG data may be pre-stored in the memory 12 instead of being supplied from an external device. In another example, at least one of the input 14 and/or the output 15 may be an external device connected to the electrocardiogram evaluation device 1 through the interface 11 or the like. In yet another example, at least some information stored in the memory 12 may be stored in an external device connected to the electrocardiogram evaluation device 1. In this case, the external device may be configured as one or more server devices.

### (3) Functional Blocks

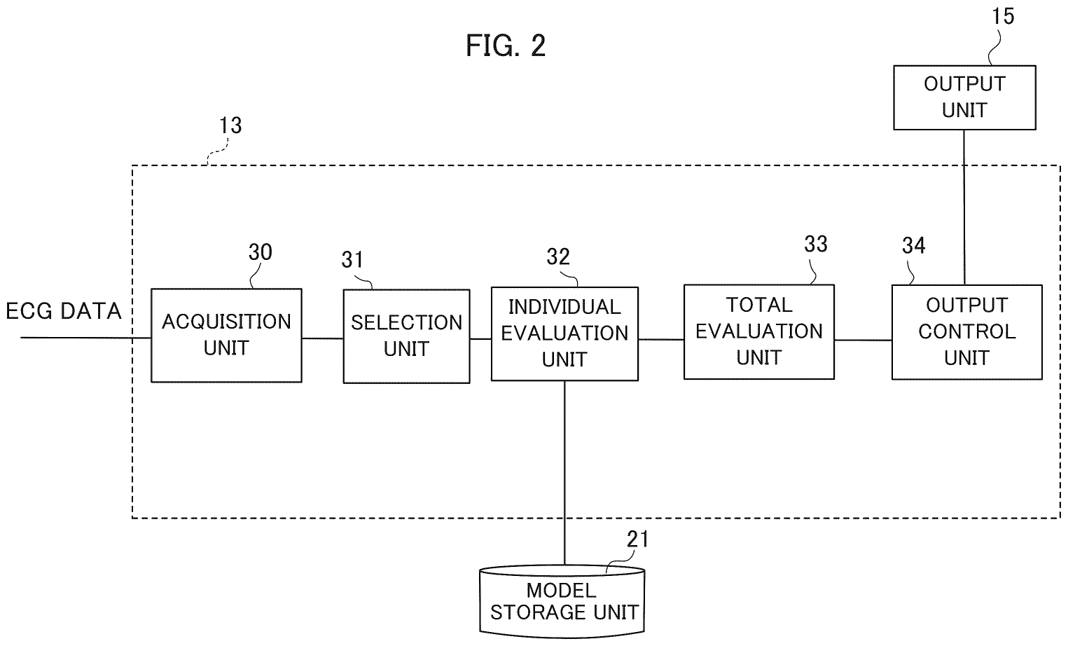

FIG. 2 is a functional block diagram of an electrocardiogram evaluation device 1. The processor 13 of the electrocardiogram evaluation device 1 functionally includes an acquisition unit 30, a selection unit 31, an individual evaluation unit 32, a total evaluation unit 33, and an output control unit 34. Here, any blocks to transmit and receive data to or from each other are connected by a solid line, but the combination of the blocks to transmit and receive data to or from each other is not limited to the combination shown in FIG. 2. The same applies to other functional block 20 diagrams described below.

The acquisition unit 30 acquires the ECG data from the external device through the interface 11. In addition, if the ECG data is either raw data of a non-quantized twelve-lead electrocardiogram or image data (including PDF file) representing signal waveforms, the acquisition unit 30 converts the data into the lead data, which is time series data quantized (including sampled and encoded) for each lead. In this case, the acquisition unit 30 may perform the above-described conversion process based on any digital conversion method or an image recognition method. The acquisition unit 30 may generate measurement data for a predetermined time extracted from the ECG data as the lead data. The predetermined time may be, for example, a time width that conforms to the input format of the model to be used. Then, the acquisition unit 30 supplies the twelve pieces of lead data corresponding to the twelve leads to the selection unit 31.

The selection unit 31 selects the lead data to be used for determination as to the presence or absence of the target disease from the twelve pieces of lead data for the twelve leads, and supplies the selected lead data to the individual evaluation unit 32. In this event, for example, if the target disease is atrial fibrillation, the selection unit 31 selects the lead data corresponding to the I lead, II lead, and V1 lead. Since the I, II and V1 leads correspond to the axes in the three-dimensional space of the heart, the electric activity of the heart can be detected spatially by using these three leads, and the presence or absence of atrial fibrillation can be accurately determined. In another example, if the target disease is cardiac enlargement, the selection unit 31 selects at least the lead data corresponding to V5 lead and V6 lead. In yet another example, if the target disease is angina pectoris, the selection unit 31 selects lead data at least corresponding to the II lead, V5 lead, and V6 lead.

In the case of evaluating a target disease specified by the input unit 14 or the like, the selection unit 31 determines leads to be used for the evaluation of the electrocardiogram regarding the specified target disease and selects the lead data corresponding to the determined leads. In this case, for example, the memory 12 or the like stores, in advance, table information indicating the correspondence between each disease as a candidate for the target disease and the leads to be used when the each disease is designated as the target disease. The “disease as a candidate for the target disease” includes any disease that is diagnosed based on an electrocardiogram. Then, the selection unit 31 refers to the table information and determines the leads to be used for the evaluation of the electrocardiogram related to the target disease. In this way, the selection unit 31 performs rule-based selection of the lead data in accordance with the target disease.

The individual evaluation unit 32 conducts an individual evaluation (also referred to as “individual evaluation”) of the electrocardiogram for each selected lead by using the lead data selected by the selection unit 31, and supplies individual evaluation information representing the individual evaluation for each lead to the total evaluation unit 33. In this case, the individual evaluation unit 32 inputs the lead data to the model configured by referring to the parameters stored in the model storage unit 21, and then acquires, as the individual evaluation, an evaluation result regarding the presence or absence (i.e., abnormal or normal) of the target disease outputted by the the model. In addition, if models are trained in advance for respective target diseases or/and for respective types of the leads, the individual evaluation unit 32 selects a model according to the target disease and the type of the lead.

The total evaluation unit 33 conducts a total evaluation (also referred to as “total evaluation”) of the electrocardiogram regarding the target disease, on the basis of the individual evaluations for respective leads calculated by the individual evaluation unit 32 and supplies the total evaluation information representing the total evaluation to the output control unit 34.

For example, for each lead, the total evaluation unit 33 takes votes of the individual evaluations (evaluation indicative of being normal or abnormal) indicated by the individual evaluation information, and determines the total evaluation by majority vote. Hereafter, the determination method of the total evaluation by majority vote is also referred to as “majority voting system”. In another example, weights are set in advance for respective leads, and the total evaluation unit 33 calculates a total score representing the probability of the presence of the target disease based on the weights for respective leads and the individual evaluations for the respective leads. For example, the higher the probability of the presence of the target disease is, the higher the calculated total score becomes. For example, the total evaluation unit 33 calculates, for each lead, the product of the confidence level of being normal and the weight, and calculates the sum of the above-described products for respective leads as the total score. The above-described confidence level may be a confidence level representing the degree of certainty for each class (in this case, a class of being normal and a class of being abnormal) that is outputted by the model that is a neural network, or may be a binarized value of the output from the model. Then, the total evaluation unit 33 determines a total evaluation indicative of being normal (i.e., absence of the target disease) if the above-described total score is smaller a predetermined threshold value. In contrasts, it determines a total evaluation indicative of being abnormal (i.e., presence of the target disease) if the total score is equal to or larger than the predetermined threshold value. The above threshold value, for example, is previously stored in the memory 12. Instead of the above-described example, the total score may be calculated to increase with decrease in the probability of the presence of the target disease. Hereafter, the method of determining the total evaluation based on the weighting and the total score is also referred to as a “score method”.

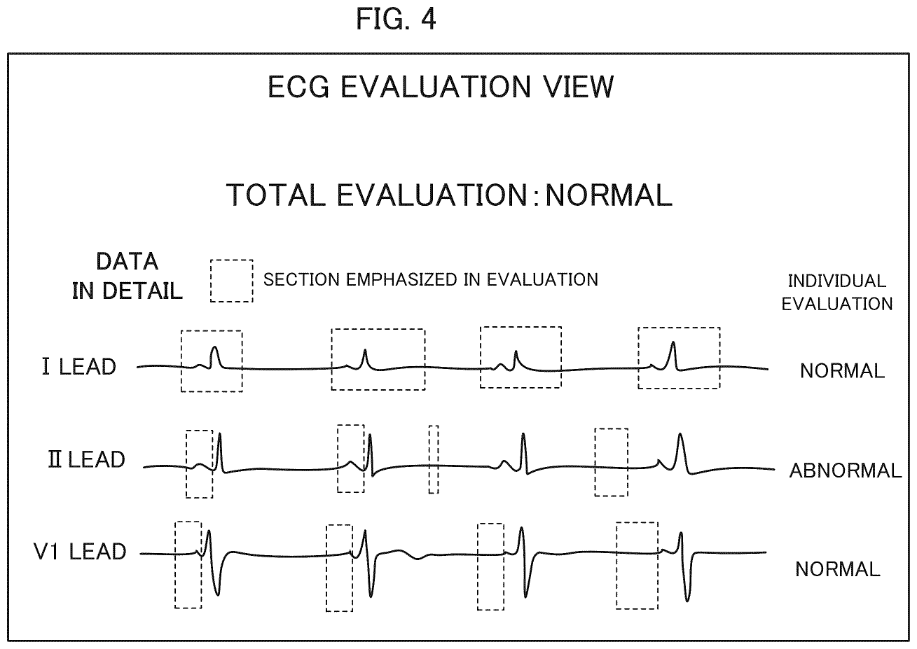

The output control unit 34 controls the output unit 15 based on the total evaluation information supplied from the total evaluation unit 33. In this case, for example, the output control unit 34 causes the output unit 15 to display the total evaluation (i.e., evaluation regarding the presence or absence of the target disease) indicated by the total evaluation information, together with the ECG data acquired by the acquisition unit 30. In some embodiments, the output control unit 34 may further display the waveform of each lead data selected by the selection unit 31 with clear indication of a section (also referred to as “section of interest”), on the above-described waveform, which is emphasized in the calculation of the individual evaluation by the model. In other words, the output control unit 34 may display the waveform of each of the selected lead data in a manner that the section of interest is clearly indicated. This display example will be described with reference to FIG. 4. The output control unit 34 functions as the “display control means”.

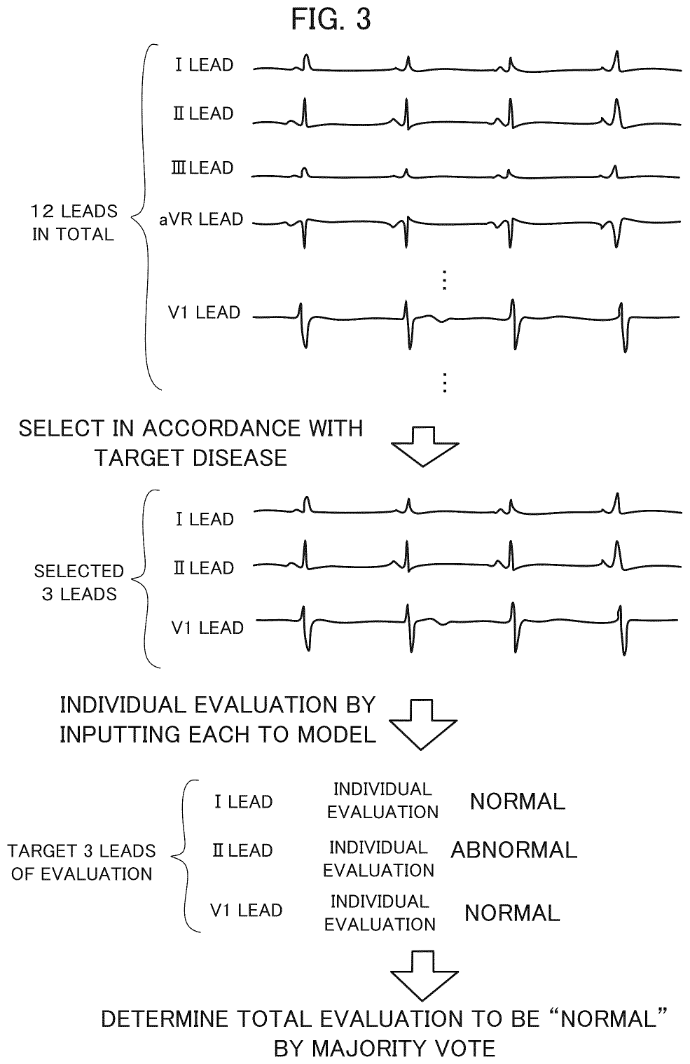

Here, a specific example will be described of the process executed by the acquisition unit 30, the selection unit 31, the individual evaluation unit 32, and the total evaluation unit 33. FIG. 3 shows the flow of the process when the target disease is atrial fibrillation.

First, the acquisition unit 30 acquires the lead data corresponding to the twelve leads. The upper part of FIG. 3 shows, as typical examples, waveforms based on the following lead data: I lead, II lead, III lead, aVR lead, and V1 lead. Then, the selection unit 31 selects, from the lead data of these twelve leads, the respective lead data of I lead, II lead, and V1 lead, which are useful for evaluating the presence or absence of atrial fibrillation which is the target disease, as shown in the middle of FIG. 3. Then, the individual evaluation unit 32 acquires the individual evaluations by inputting respective pieces of lead data corresponding to the I lead, II lead, and V1 lead to the model. Here, the individual evaluation unit 32 generates the individual evaluation information indicating that the individual evaluation based on the lead data of the I-lead is “normal”, the individual evaluation based on the lead data of the II lead is “abnormal”, and the individual evaluation based on the V1 lead is “normal”, and supplies the individual evaluation information to the total evaluation unit 33. Then, based on the individual evaluation information, the total evaluation unit 33 determines the majority of the individual evaluations by taking a vote, and determines the total evaluation to be the evaluation “normal” by majority vote. The total evaluation unit 33 may determine the total evaluation according to the score method instead of the majority voting method.

FIG. 4 is an example of an electrocardiogram evaluation screen image that is displayed on the output unit 15 by the output control unit 34. The output control unit 34 generates a display signal based on various kinds of information supplied from other processing units and supplies the display signal to the output unit 15, thereby causing the output unit 15 to display the electrocardiogram evaluation screen image.

In this case, the output control unit 34 displays the total evaluation based on the total evaluation information generated by the total evaluation unit 33, and displays the waveform of the lead data selected by the selection unit 31. The output control unit 34 clarifies the section of interest, which is emphasized in the calculation of the individual evaluation by the model, on the waveform. Here, the output control unit 34 highlights the section of interest described above using a broken line frame. Thus, the output control unit 34 can present a portion, in the electrocardiogram, on which the evaluation is based and suitably support the examiner to determine the final diagnosis result.

Here, a specific example of the determination method of the section of interest will be supplemented. For example, when the model is a convolutional neural network, the electrocardiogram evaluation device 1 may add an attention mechanism to the model so that output data prior to the full connection layer is inputted to the attention mechanism, and identify the section of interest to be any section in which a coefficient outputted by the attention mechanism is equal to or larger than a predetermined value.

Each component of the acquisition unit 30, the selection unit 31, the individual evaluation unit 32, the total evaluation unit 33 and the output control unit 34 can be realized by the processor 13 executing a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

### (4) Processing Flow

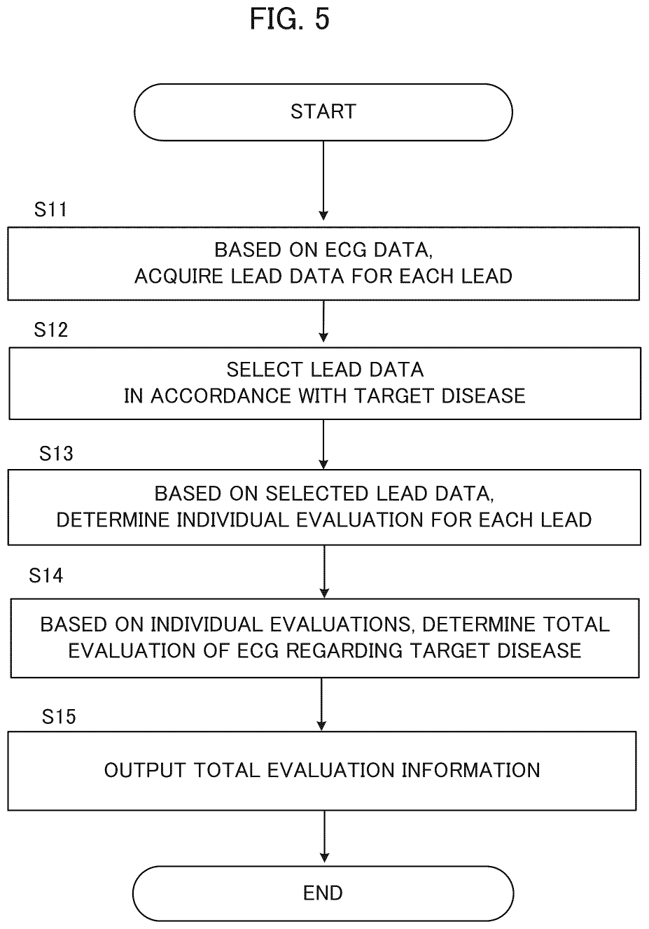

FIG. 5 is an example of a flowchart illustrating an outline of the process performed by the electrocardiogram evaluation device 1 in the first example embodiment.

First, the electrocardiogram evaluation device 1 acquires lead data for each lead based on ECG data supplied from an external device (step S11). In this case, for example, the electrocardiogram evaluation device 1 applies a digitization process to the acquired ECG data to generate lead data that is numerical data in time series.

Then, the electrocardiogram evaluation device 1 selects the lead data according to the target disease (step S12). In this case, for example, based on the input information by the input unit 14 or the setting information stored in the memory 12, the electrocardiogram evaluation device 1 recognizes the target disease, and selects the lead data related to the target disease on a rule basis.

Next, the electrocardiogram evaluation device 1 determines an individual evaluation for each lead based on the selected lead data (step S13). In this case, the electrocardiogram evaluation device 1 inputs each piece of the selected lead data into the model configured by referring to the model storage unit 21 and then acquires, from the model, each individual evaluation which individually indicates the evaluation regarding the presence or absence of the target disease for each lead.

Then, the electrocardiogram evaluation device 1 determines the total evaluation of the electrocardiogram regarding the target disease based on the individual evaluations calculated at step S13 (step S14). Each individual evaluation corresponds to a provisional evaluation of the electrocardiogram, and the total evaluation corresponds to the final evaluation of the electrocardiogram determined by the electrocardiogram evaluation device 1.

Then, the electrocardiogram evaluation device 1 outputs the total evaluation information representing the total evaluation (step S15). In this case, the electrocardiogram evaluation device 1 may store the total evaluation information in the memory 12 in place of or in addition to outputting the total evaluation information to the output unit 15, or may transmit the total evaluation information to an external device.

### Second Example Embodiment

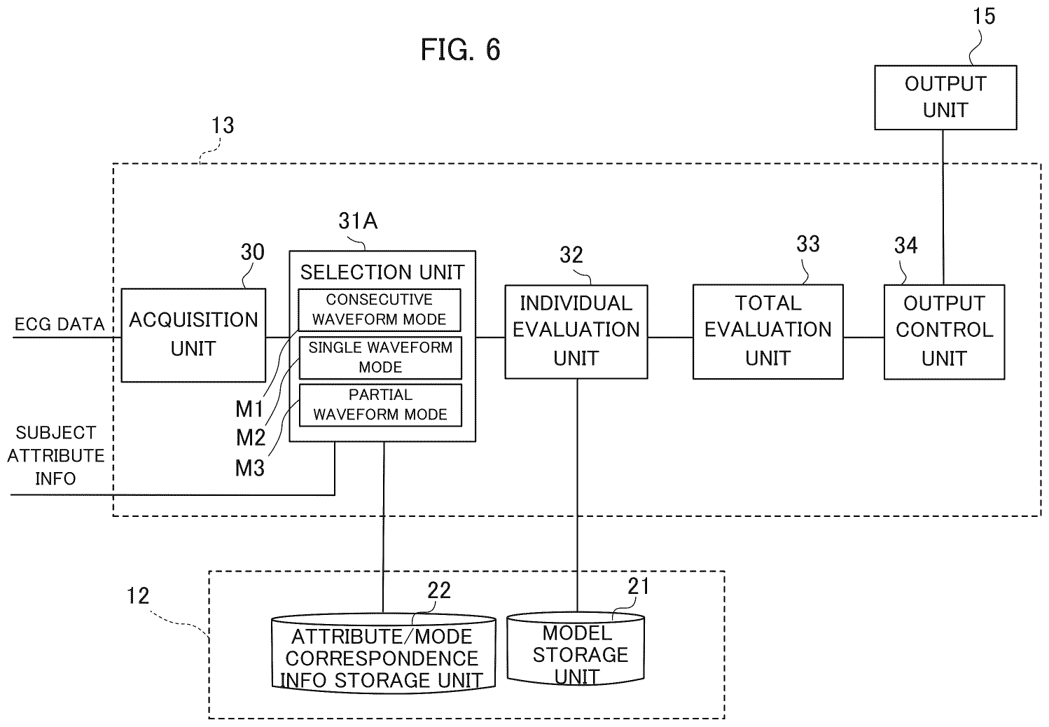

FIG. 6 is a functional block of the electrocardiogram evaluation device 1 according to the second example embodiment. The electrocardiogram evaluation device 1 according to the second example embodiment differs from the electrocardiogram evaluation device 1 according to the first example embodiment in that it further performs processing for extracting, based on the attribute information regarding the subject, data to be used for determination of the evaluation of the electrocardiogram from the selected lead data. Hereinafter, the same components as those in the first example embodiment are appropriately denoted by the same reference numerals, and a description thereof will be omitted.

The selection unit 31A performs not only the process of selecting the lead data related to the target disease but also the process of extracting target data (also referred to as “evaluation target data”) of usage for determining the evaluation of the electrocardiogram from the selected lead data, on the basis of the attribute information regarding the subject and the attribute/mode correspondence information stored in advance in the attribute/mode correspondence information storage unit 22. The evaluation target data is data to be inputted to the model.

In the process of extracting the evaluation target data, the selection unit 31A selectively executes any one of three waveform selection modes (consecutive waveform mode M1, single waveform mode M2, and partial waveform mode M3). Here, the consecutive waveform mode M is a model, as in the first example embodiment, to determine that the evaluation target data is the data (i.e., the whole lead data) of the consecutive waveforms (four waveforms in the example embodiment shown in FIG. 3). The single waveform mode M2 is a mode to determine that the evaluation target data is lead data corresponding to a single waveform (unit waveform), and the partial waveform mode M3 is a mode to determine that the evaluation target data is partial lead data corresponding to a partial waveform smaller than a single waveform. The consecutive waveform mode M1 is an example of the “first mode”, the single waveform mode M2 is an example of the “second mode”, the partial waveform mode M3 is an example of the “third mode”.

Here, the attribute information regarding the subject is information indicating any attribute of the subject affected in the electrocardiogram examination, and examples of such attribute include age, gender, and past medical history. The selection unit 31A may receive the attribute information regarding the subject from an external device, which manages the attribute information regarding the subject, through the interface 11. In another example, the selection unit 31A may generate the attribute information based on the output from the input unit 14 based on the user operation, or may acquire the attribute information from the memory 12 which stores the attribute information in advance.

The attribute/mode correspondence information is information in which assumed subject's attributes are associated with the waveform selection modes suitable for the respective attributes. The attribute/mode correspondence information is generated in advance based on electrocardiogram examination results or the like conducted in advance, and is stored in the attribute/mode correspondence information storage unit 22.

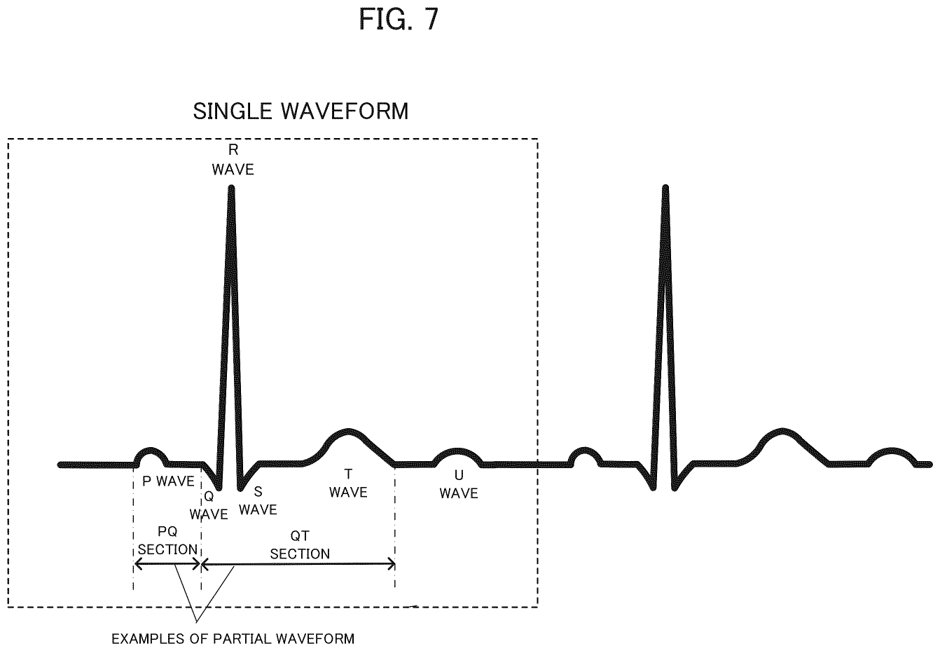

FIG. 7 is an enlarged view of the waveform of lead data. As an example, the lead data includes two waveforms (two pieces of waveform), one of which is enclosed by a dashed frame. As shown, each waveform includes P wave, Q wave, R wave, S wave, T wave, U wave and the like, it is possible to detect the positions of these waves by signal processing.

Then, in using the consecutive waveform model M1, the selection unit 31A determines that the evaluation target data is the whole lead data indicating the consecutive waveform. Further, in using the single waveform mode M2, the selection unit 31A determines that the evaluation target data is data representing a single waveform extracted from the lead data. Further, in using the partial waveform mode M3, the selection unit 31A determines that the evaluation target data is data representing a partial waveform extracted from the lead data. For example, the evaluation target data in the partial waveform mode M3 may be data representing the section (PQ section) from the P wave to the Q wave, the section (QT section) from the Q wave to the T wave, or any other section between any two waves, or may be data representing a section (e.g., P wave section, R wave section) obtained by cutting off only an individual wave.

Here, in using the single waveform mode M2, the selection unit 31A may extract evaluation target data corresponding to any one waveform among plural waveforms included in the target lead data. In another example, the selection unit 31A may extract evaluation target data corresponding to each of the plural waveforms. In the latter case, for example, the individual evaluation unit 32 determines the individual evaluation on the target lead data, based on the evaluation result from the model obtained by inputting the evaluation target data corresponding to each of the plural waveforms to the model. In this case, for example, the individual evaluation unit 32 may determine the individual evaluation described above, based on the majority voting method. In using the partial waveform mode M3, the selection unit 31A also extracts data corresponding to a part of at least one waveform as evaluation target data. Then, if plural pieces of evaluation target data corresponding to respective parts of plural waveforms are generated, the individual evaluation unit 32 determines the individual evaluation of the target lead data, based on the evaluation results from the model obtained by inputting each of the plural pieces of evaluation target data to the model.

The models used in the consecutive waveform mode M1, the single waveform mode M2, and the partial waveform mode M3 may be different from one another. In this instance, the learned parameters of the models to be used in the consecutive waveform mode M1, the single waveform mode M2, and the partial waveform mode M3 are previously stored in the model storage unit 21.

Then, the selection unit 31A selects the waveform selection mode associated with the attribute indicated by the attribute information of the subject with reference to the attribute/mode correspondence information. The selection unit 31A supplies the evaluation target data, which is extracted from each piece of lead data based on the selected waveform selection mode, to the individual evaluation unit 32. The record corresponding to the partial waveform mode M3 in the attribute/mode correspondence information further includes information for identifying a section (e.g., the PQ section, the QT section, or any other wave section) of the partial waveform to be extracted as the evaluation target data. Therefore, even when the partial waveform mode M3 is selected, the selection unit 31A can extract the evaluation target data based on the attribute/mode correspondence information. In this way, the selection unit 31A accurately extracts the evaluation target data required for the evaluation based on the attribute of the subject.

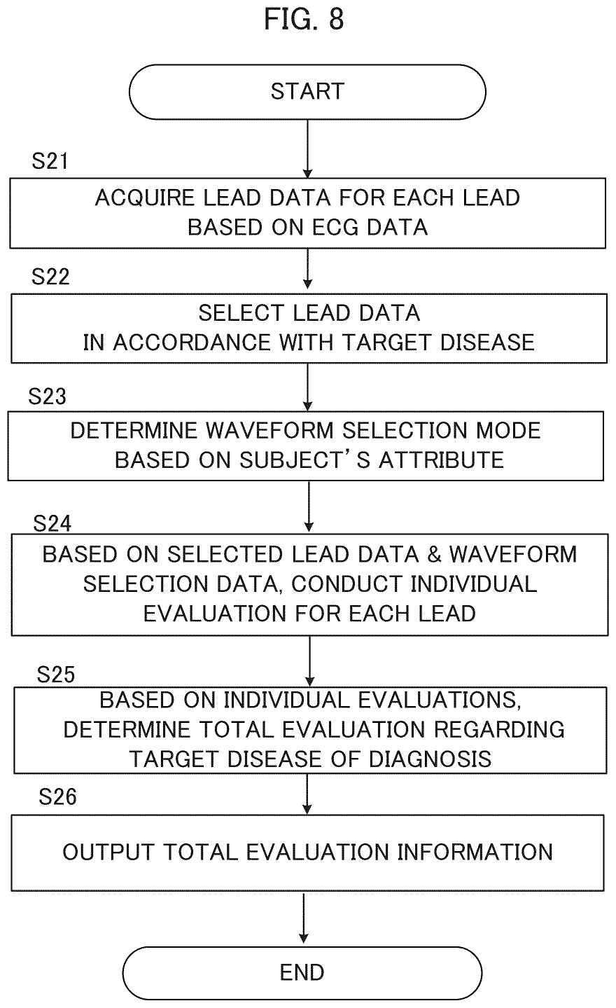

FIG. 8 is an example of a flowchart illustrating an outline of the process performed by the electrocardiogram evaluation device 1 in the second example embodiment.

First, the electrocardiogram evaluation device 1 acquires lead data for each lead based on ECG data supplied from an external device (step S21). Then, the electrocardiogram evaluation device 1 selects one or more pieces of lead data according to the target disease (step S22).

Next, based on the attribute of the subject, the electrocardiogram evaluation device 1 determines the waveform selection mode (step S23). In this case, based on the attribute information regarding the subject supplied from the external device and the attribute/mode correspondence information stored in the attribute/mode correspondence information storage unit 22, the electrocardiogram evaluation device 1 determines the waveform selection mode according to the attribute of the subject. If the waveform selection mode corresponding to the attribute of the subject is not recorded in the attribute/mode correspondence information, the electrocardiogram evaluation device 1 sets the waveform selection mode to be the consecutive waveform mode M1, for example.

Then, the electrocardiogram evaluation device 1 determines the individual evaluation for each lead based on the selected lead data and the determined waveform selection mode (step S24). In this case, the electrocardiogram evaluation device 1 inputs, to the model configured by referring to the model storage unit 21, data obtained by cutting off a section determined by the waveform selection mode from the selected lead data as evaluation target data. Then, based on the evaluation results obtained from the model in response to the input, the electrocardiogram evaluation device 1 determines the individual evaluation for every lead which individually indicates the evaluation regarding presence or absence of the target disease.

Then, the electrocardiogram evaluation device 1 determines the total evaluation of the electrocardiogram regarding the target disease, based on the individual evaluations calculated at step S24 (step S25). Then, the electrocardiogram evaluation device 1 outputs the total evaluation information representing the total evaluation (step S26).

The electrocardiogram evaluation device 1 according to the second example embodiment determine an appropriate waveform selection mode according to the attribute of the subject, thereby generating individual evaluations which individually indicate the evaluations regarding the presence or absence of the target disease with a high degree of accuracy and outputting an accurate evaluation result on the target disease.

### Third Example Embodiment

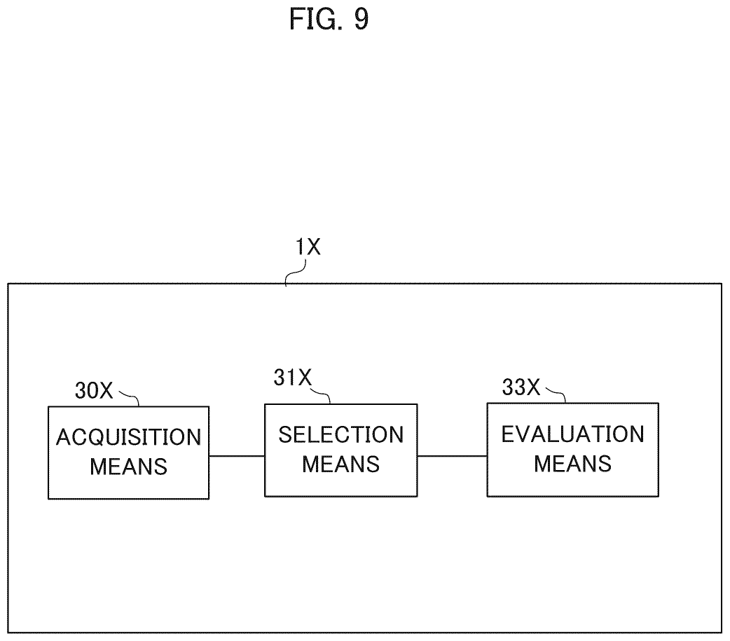

FIG. 9 is a block diagram of the electrocardiogram evaluation 1X in the third example embodiment. The electrocardiogram evaluation device 1X mainly includes an acquisition means 30X, a selection means 31X, and an evaluation means 33X. The electrocardiogram evaluation device 1X may be configured by a plurality of devices.

The acquisition means 30X is configured to acquire electrocardiogram (ECG) data regarding an electrocardiogram of a subject. Examples of the acquisition means 30X include the acquisition unit 30 in the first example embodiment or the second example embodiment. The selection means 31X is configured to select, from the electrocardiogram data, lead data of one or more leads corresponding to a target disease of examination. Examples of the selection means 31X include the selection unit 31 in the first example embodiment and the selection unit 31A in the second example embodiment. The evaluation means 33X is configured to evaluate the electrocardiogram regarding the target disease based on the selected lead data. Examples of the evaluation means 33X include the individual evaluation unit 32 and the total evaluation unit 33 in the first example embodiment or the second example embodiment.

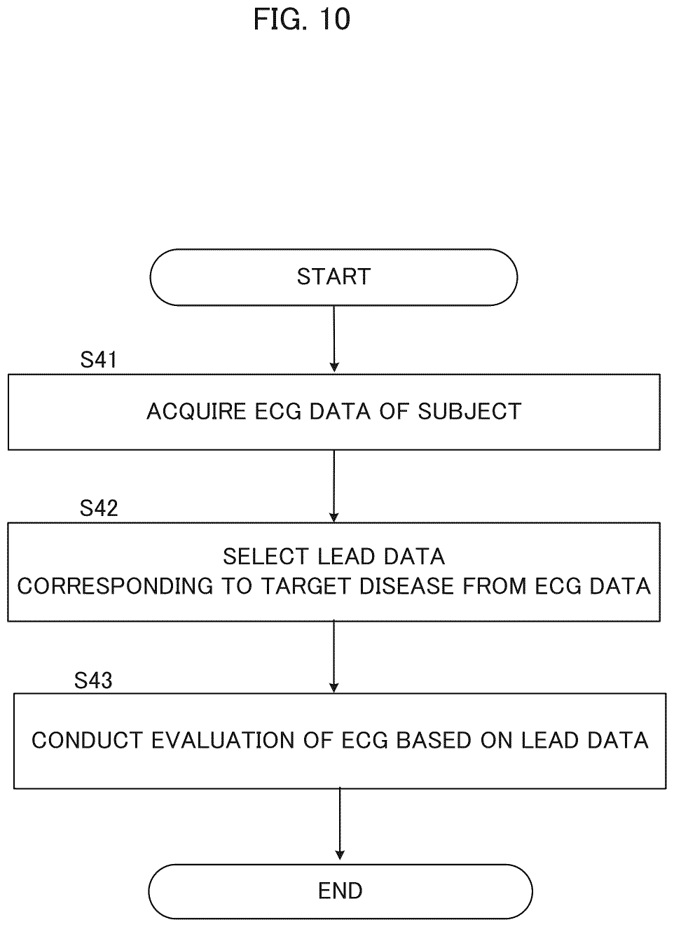

FIG. 10 is an example of the flowchart illustrating a process performed by the electrocardiogram evaluation 1X in the third example embodiment. The acquisition means 30X acquires electrocardiogram data regarding an electrocardiogram of a subject (step S41). The selection means 31X selects, from the electrocardiogram data, lead data of one or more leads corresponding to a target disease of examination (step S42). The evaluation means 33X evaluates the electrocardiogram regarding the target disease based on the selected lead data (step S43).

According to the third example embodiment, using the lead data related to the target disease of examination, the electrocardiogram evaluation device 1X can accurately conduct the electrocardiogram evaluation on the target disease.

In the example embodiments described above, the program is stored by any type of a non-transitory computer-readable medium (non-transitory computer readable medium) and can be supplied to a processor or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### [Supplementary Note 1]

An electrocardiogram evaluation device comprising:
an acquisition means configured to acquire electrocardiogram data regarding an electrocardiogram of a subject;
a selection means configured to select, from the electrocardiogram data, lead data of one or more leads corresponding to a target disease of examination; and
an evaluation means configured to evaluate the electrocardiogram regarding the target disease based on the selected lead data.

### [Supplementary Note 2]

The electrocardiogram evaluation device according to Supplementary Note 1,
the evaluation means comprises:
   an individual evaluation means configured to determine an individual evaluation, which is an evaluation for each of the leads, based on the lead data for each of the leads, and
   a total evaluation determination means configured to determine a total evaluation into which the individual evaluations for the leads are synthesized.

### [Supplementary Note 3]

The electrocardiogram evaluation device according to Supplementary Note 2,
wherein the total evaluation determination means is configured to determine the total evaluation based on a majority vote of the individual evaluations.

### [Supplementary Note 4]

The electrocardiogram evaluation device according to Supplementary Note 2,
wherein the total evaluation determination means is configured to
   calculate a score based on weights set for the respective leads and the individual evaluations and
   determine the total evaluation based on the score.

### [Supplementary Note 5]

The electrocardiogram evaluation device according to any one of Supplementary Notes 1 to 4, further comprising
a display control means configured to display a waveform based on the lead data on a display device in a manner that a section of interest in the evaluation is indicated on the waveform.

### [Supplementary Note 6]

The electrocardiogram evaluation device according to any one of Supplementary Notes 1 to 5,
wherein the selection means is configured to select the lead data corresponding to I lead, II lead, and V1 lead if the target disease is atrial fibrillation.

### [Supplementary Note 7]

The electrocardiogram evaluation device according to any one of Supplementary Notes 1 to 6,
wherein the selection means is configured to perform any one of:
   a first mode to set a whole of the lead data as evaluation target data, which is the selected lead data to be used for determining the evaluation;
   a second mode to extract data corresponding to a unit waveform of the lead data as the evaluation target data; and
   a third mode to extract data corresponding to a partial waveform of the lead data as the evaluation target data.

### [Supplementary Note 8]

The electrocardiogram evaluation device according to any one of Supplementary Notes 1 to 7,
wherein the selection means is configured to extract, based on attribute information regarding the subject, evaluation target data, which is data to be used for determining the evaluation, from the selected lead data.

### [Supplementary Note 9]

An electrocardiogram evaluation method executed by the computer, the electrocardiogram evaluation method comprising:
acquiring electrocardiogram data regarding an electrocardiogram of a subject;
selecting, from the electrocardiogram data, lead data of one or more leads corresponding to a target disease of examination; and
evaluating the electrocardiogram regarding the target disease based on the selected lead data.

### [Supplementary Note 10]

A storage medium storing a program executed by a computer, the program causing the computer to:
acquire electrocardiogram data regarding an electrocardiogram of a subject;
select, from the electrocardiogram data, lead data of one or more leads corresponding to a target disease of examination; and
evaluate the electrocardiogram regarding the target disease based on the selected lead data.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. Each example embodiment can be appropriately combined with other example embodiments. All Patent and Non-Patent Literatures mentioned in this specification are incorporated by reference in its entirety.

### DESCRIPTION OF REFERENCE NUMERALS

1, 1X electrocardiogram Evaluation Device
11 Interface
12 Memory
13 Processor
14 Input unit
15 Output unit
21 Model storage unit
22 Attribute/mode correspondence information storage unit

What is claimed is:

1. An electrocardiogram evaluation device comprising:
at least one memory configured to store instructions; and
at least one processor configured to execute the instructions to:
acquire electrocardiogram data regarding an electrocardiogram of a subject;
select, from the electrocardiogram data, lead data of one or more leads corresponding to a target disease of examination;
evaluate the electrocardiogram regarding the target disease based on the selected lead data; and
display a waveform based on the lead data on a display device in a manner that a section of interest in the evaluation is indicated on the waveform.

2. The electrocardiogram evaluation device according to claim 1,
the at least one processor is configured to execute the instructions
to determine an individual evaluation, which is an evaluation for each of the leads, based on the lead data for each of the leads, and
to determine a total evaluation into which the individual evaluations for the leads are synthesized.

3. The electrocardiogram evaluation device according to claim 2,
wherein the at least one processor is configured to execute the instructions to determine the total evaluation based on a majority vote of the individual evaluations.

4. The electrocardiogram evaluation device according to claim 2,
wherein the at least one processor is configured to execute the instructions to
calculate a score based on weights set for the respective leads and the individual evaluations and
determine the total evaluation based on the score.

5. (canceled)

6. The electrocardiogram evaluation device according to claim 1,
wherein the at least one processor is configured to execute the instructions selection means is configured to select the lead data corresponding to I lead, II lead, and V1 lead if the target disease is atrial fibrillation.

7. The electrocardiogram evaluation device according to claim 1,
wherein the at least one processor is configured to execute the instructions to perform any one of:
a first mode to set a whole of the lead data as evaluation target data, which is the selected lead data to be used for determining the evaluation;
a second mode to extract data corresponding to a unit waveform of the lead data as the evaluation target data; and
a third mode to extract data corresponding to a partial waveform of the lead data as the evaluation target data.

8. The electrocardiogram evaluation device according to claim 1,
wherein the at least one processor is configured to execute the instructions to extract, based on attribute information regarding the subject, evaluation target data, which is data to be used for determining the evaluation, from the selected lead data.

9. An electrocardiogram evaluation method executed by a computer, the electrocardiogram evaluation method comprising:
acquiring electrocardiogram data regarding an electrocardiogram of a subject;
selecting, from the electrocardiogram data, lead data of one or more leads corresponding to a target disease of examination; and
evaluating the electrocardiogram regarding the target disease based on the selected lead data.

10. A storage medium storing a program executed by a computer, the program causing the computer to:
acquire electrocardiogram data regarding an electrocardiogram of a subject;
select, from the electrocardiogram data, lead data of one or more leads corresponding to a target disease of examination; and
evaluate the electrocardiogram regarding the target disease based on the selected lead data.

11. The electrocardiogram evaluation device according to claim 1,
wherein the at least one processor is configured to execute the instructions to evaluate the electrocardiogram using a model, the model being subjected to machine learning to output an evaluation result regarding a presence or absence of the target disease when the lead data is inputted to the model.

12. The electrocardiogram evaluation device according to claim 1,
wherein the at least one processor is configured to execute the instructions to display the waveform to assist an examiner's decision making regarding a diagnosis.
